(19) United States
(12) Patent Application Publication
(10) Pub. No.: US 2025/0318709 A1
(43) Pub. Date: Oct. 16, 2025

(54) SUCTION VALVE ACTUATORS FOR ENDOSCOPES

(71) Applicant: Boston Scientific Scimed, Inc., Maple Grove, MN (US)

(72) Inventors: Rosangel Aimee Ramos Espinoza, Allston, MA (US); Ryan Vincent William Pollock, Sterling, MA (US)

(73) Assignee: Boston Scientific Scimed, Inc., Maple Grove, MN (US)

(21) Appl. No.: 19/173,041

(22) Filed: Apr. 8, 2025

Related U.S. Application Data
(60) Provisional application No. 63/634,247, filed on Apr. 15, 2024.

(51) Int. Cl. A61B 1/00

(52) U.S. Cl. CPC A61B 1/00068

(57) ABSTRACT

A suction valve for endoscopes (e.g., duodenoscopes) includes a valve element that rotates between open and closed positions in response to linear movement of a plunger. In some examples, a meshing gear arrangement provides the mechanical interaction between the plunger and the valve element. In some examples, the valve element's rotation is by virtue of at least portions of the valve element being resiliently flexible. In some examples, the valve element's resilience urges the plunger to its relaxed home position. In some examples, the axis about which the valve element rotates is parallel to the plunger's linear travel path. In some examples, the axis about which the valve element rotates is perpendicular to the plunger's linear travel path.

### CROSS-REFERENCE TO RELATED APPLICATIONS

This application claims the benefit of U.S. Provisional Application No. 63/634,247, filed Apr. 15, 2024, the disclosure of which is incorporated herein by reference in its entirety.

### TECHNICAL FIELD

Various aspects of this disclosure relate generally to endoscopes (e.g., duodenoscopes, colonoscopes, bronchoscopes, etc.) and more specifically to suction valves for endoscopes.

### BACKGROUND

Endoscopes enable medical practitioners to directly visualize internal cavities of patients without the need for invasive surgeries. Among the various types of endoscopes, duodenoscopes hold a prominent place due to their capability to explore the upper gastrointestinal tract, particularly the duodenum, pancreas, and bile ducts. Duodenoscopes facilitate not only visual examinations but also a range of therapeutic procedures, making them indispensable tools in modern medicine.

Duodenoscopes typically comprise a flexible tubular probe extending from a handle body. The probe is inserted into the patient, while the operator holds the handle body. A light source at a distal end of the probe provides illumination for viewing. Often a high-resolution camera is adjacent to the light source for capturing real-time images or videos of the internal cavities.

A duodenoscope's probe usually includes an internal working channel, allowing for the insertion of various instruments for procedures like biopsies, tissue removal, or stent placement. To provide maneuverability and access to intricate anatomical structures, many duodenoscope probes have internal wires. The tension in the wires can be adjusted in opposing sets of two by manipulating knobs on a handle body of the duodenoscope. Adjusting the wire tension enables bending and steering of the probe.

Handle bodies typically include a bifluidic valve (air/water valve) and a suction valve, which the operator controls manually. Bifluidic valves can be used for controlling fluid flow, such as air for insufflation and water for irrigation. Insufflation is a technique involving the introduction of air or carbon dioxide into the cavity being examined, which helps to expand the space, allowing for better visibility of the targeted area.

Irrigation typically involves the introduction of liquids, such as sterile water or saline. Irrigation can help clear blood, debris, or mucus from the visual field, ensuring clear visibility. Irrigation can also aid in therapeutic interventions by flushing out areas of interest, allowing for better access and manipulation of tissues.

Suction valves on the handle body are used by the operator to control the amount of suction at the distal end of the probe. Suction valves often include a valve spool that can be moved manually between open and closed positions. When open, the applied suction can clear the probe's field of view by drawing fluid, tissue and other matter back through a suction tube within the probe. Moving the valve spool to the closed position terminates the suction. Suction valves typically include a spring for urging the valve spool to its closed position.

### SUMMARY

The present disclosure generally pertains to suction valves for controlling a fluid flowing through an endoscope that includes a flexible tubular probe extending from a handle body. In some examples, the endoscope is connectable to a suction source. In some examples, the suction valve is configurable selectively to a suction configuration and a vented configuration. Some examples of the suction valve include a valve housing supported by the handle body. In some examples, the valve housing defines a first opening and a second opening. Some examples of the suction valve include a valve element within the valve housing. In some examples, the valve element includes a sealing portion that is rotatable about a first axis between an open position and a closed position relative to the valve housing. Some examples of the suction valve include a plunger engaging the valve element and being movable relative thereto. In some examples, the plunger is further movable linearly along a second axis between a home position and a depressed position relative to the valve housing. In some examples, the sealing portion moves from the closed position to the open position in response to the plunger moving from the home position to the depressed position. In some examples, the sealing portion provides a greater obstruction to fluid communication between the first opening and the second opening when the sealing portion is in the closed position than when the sealing portion is in the open position.

In some examples, a suction valve includes a valve housing to be supported by the handle body. In some examples, the valve housing defines a first opening and a second opening. Some examples of the suction valve include a valve element within the valve housing. In some examples, the valve element includes a sealing portion that is rotatable about a first axis between an open position and a closed position relative to the valve housing. In some examples, the valve element defines a bore encircling the first axis. Some examples of the suction valve include a plunger extending at least partially through the bore of the valve element. In some examples, the plunger is movable linearly along a second axis between a home position and a depressed position relative to the valve housing. In some examples, the second axis is substantially parallel with the first axis. In some examples, the sealing portion rotates about the first axis from the closed position to the open position in response to the plunger moving from the home position to the depressed position. In some examples, the sealing portion provides a greater obstruction to fluid communication between the first opening and the second opening when the sealing portion is in the closed position than when the sealing portion is in the open position.

In some examples, a suction valve includes a valve housing to be supported by the handle body. In some examples, the valve housing defines a first opening and a second opening. Some examples of the suction valve include a valve element within the valve housing. In some examples, the valve element includes a lever and a flap with a sealing portion on the flap. In some examples, the lever and the flap are a seamless integral portion of the valve element. In some examples, the sealing portion is rotatable about a first axis between an open position and a closed position relative to the valve housing. Some examples of the suction valve include a plunger engaging the lever and being movable linearly along a second axis between a home position and a depressed position relative to the valve housing. In some examples, the second axis is substantially perpendicular to the first axis. In some examples, the valve element pivots by resilient deflection about the first axis from the closed position to the open position in response to the plunger moving along the second axis from the home position to the depressed position. In some examples, the sealing portion provides a greater obstruction to fluid communication between the first opening and the second opening when the sealing portion is in the closed position than when the sealing portion is in the open position.

The preceding summary is provided to facilitate an understanding of some of the features of the present disclosure and is not intended to be a full description. A full appreciation of the disclosure can be gained by taking the entire specification, claims, drawings and abstract as a whole.

### BRIEF DESCRIPTION OF THE DRAWINGS

FIG. 1 is a perspective view of an example endoscope with a suction valve incorporating various aspects of the present disclosure.

FIG. 2 is a cross-sectional view schematically illustrating an example suction valve for the endoscope shown in FIG. 1, wherein a pushbutton of the suction valve is in a home position.

FIG. 3 is a cross-sectional view schematically illustrating an example suction valve for the endoscope shown in FIG. 1, wherein a pushbutton of the suction valve is in a depressed position.

FIG. 4 is a perspective view schematically illustrating a portion of an example suction valve for the endoscope shown in FIG. 1.

FIG. 5 is a cross-sectional view schematically illustrating an example suction valve for the endoscope shown in FIG. 1, wherein a pushbutton of the suction valve is in its home position.

FIG. 6 is a cross-sectional view of an example suction valve for an endoscope, such as the one shown in FIG. 1.

FIG. 7 is a cross-sectional view similar to FIG. 6 but smaller, wherein the suction valve is in a vented configuration.

FIG. 8 is a cross-sectional view similar to FIG. 7 but showing the suction valve in a suction configuration.

FIG. 9 is a top view of the suction valve shown in FIG. 7.

FIG. 10 is a cross-sectional view taken along line 10-10 of FIG. 7.

FIG. 11 is a cross-sectional view taken along line 11-11 of FIG. 8.

FIG. 12 is an exploded assembly view of the suction valve shown in FIGS. 6-11.

FIG. 13 is a cross-sectional view taken along line 13-13 of FIG. 7.

FIG. 14 is an exploded assembly view of an alternate example of a plunger and a valve element for use in a suction valve, such as the one shown in FIGS. 6-11.

FIG. 15 is a cross-sectional view similar to FIG. 13 but showing the alternate example plunger and valve element of FIG. 14.

FIG. 16 is a cross-sectional view similar to FIG. 6 but showing another example suction valve for an endoscope, such as the endoscope shown in FIG. 1.

FIG. 17 is a cross-sectional view similar to FIG. 16 but smaller, wherein the suction valve is in a vented configuration.

FIG. 18 is a cross-sectional view similar to FIG. 17 but showing the suction valve in a suction configuration.

FIG. 19 is a top view of a valve element used in the suction valve shown in FIG. 17, wherein the valve element is in a closed position.

FIG. 20 is a top view similar to FIG. 19, but showing the valve element in an open configuration, in the same shape it would assume in FIG. 18.

FIG. 21 is a cross-sectional view similar to FIG. 17 but with the addition of an example leaf spring urging the valve element to the closed position.

FIG. 22 is a cross-sectional view similar to FIG. 21 but showing the valve element in the open position.

FIG. 23 is a cross-sectional view similar to FIG. 21 but with the leaf spring being a seamless integral part of the valve element.

FIG. 24 is a cross-sectional view similar to FIG. 23 but showing the valve element in the open position.

### DESCRIPTION

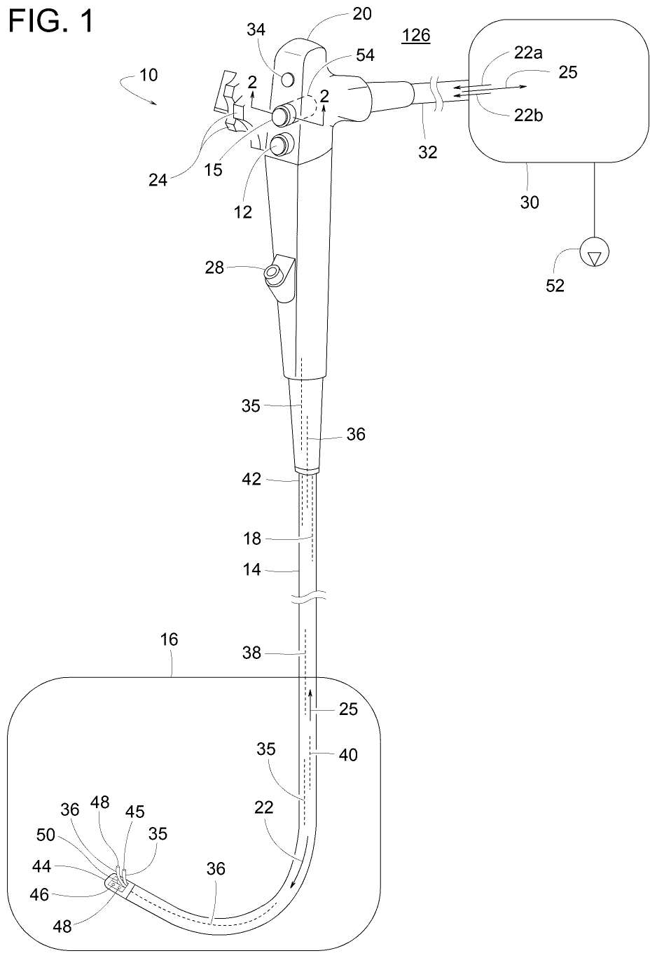

FIGS. 1-4 show various examples of a suction valve 15 (e.g., suction valves 15a and 15b) for an endoscope 10 and methods for using them. The term, “endoscope” represents any medical apparatus with a flexible tubular probe 14 for inserting into a patient 16 to visually explore the patient's internal tissues and cavities and to introduce or withdraw water, air, or other fluids when desired. Some example endoscopes 10 have internal wires 18 with adjustable tension for bending and steering the flexible tubular probe 14. Some examples of the endoscope 10, shown in FIG. 1, include duodenoscopes, colonoscopes, ureteroscopes, bronchoscopes, laparoscopes, sheaths, and catheters.

The endoscope 10 is illustrated as an example, so many of the following listed components are optional. Some examples of the endoscope 10 include components such as a handle body 20, the flexible tubular probe 14 extending from the handle body 20, the suction valve 15 for controlling the suction of a fluid 25 (e.g., bodily fluids) drawn back through the flexible tubular probe 14, a bifluidic valve 12 (air/water valve) for controlling the flow of a fluid 22 (e.g., a liquid 22a and a gas 22b), steering knobs 24 to adjust the tension in the internal wires 18, a biopsy port 28 for sampling withdrawn tissue or fluid, a control unit 30, an umbilicus 32 connecting the control unit 30 to the handle body 20, and an image capture button 34.

The control unit 30 and/or its associated external components provide various functions, such as supplying liquid 22a (e.g., water, saline, etc.), supplying gas 22b (e.g., air, carbon dioxide, etc.), providing a source of vacuum 52 (e.g., a vacuum pump, venturi, etc.), sending and receiving electrical signals, processing electrical signals, etc. Some of these functions are optional. The umbilicus 32 connects the control unit 30 in signal communication or fluid communication with the bifluidic valve 12, the suction valve 15, the flexible tubular probe 14, or other endoscope-related components. The term, “vacuum” means anything less than atmospheric pressure (i.e., less than 14.7 psia).

In some examples, the flexible tubular probe 14 contains various components such as the internal wires 18 for steering, tubing 36 (one or more tubes) for conveying fluids 22, a suction tube 35 for drawing the fluid 25 from the patient 16, a fiber optic cable 38 for conveying images or light, and electrical wires 40 for conveying electrical power or signals. Some of these probe components are optional.

The flexible tubular probe 14 has a proximal end 42 and a distal end 44. The proximal end 42 connects to the handle body 20, and the distal end 44 extends away from the handle body 20. At the distal end 44, some examples of the flexible tubular probe 14 include a light 46 (or fiber optic cable leading thereto) for illuminating a patient's internal cavities, a camera 47 (or fiber optic cable leading thereto), a tip 45 of the suction tube 35, a tip 48 of the tubing 36, and an elevator 50 for tilting tips 45 and/or 48. The elevator 50 is also known as a swing stand, a pivot stand, and a raising bed.

The tip 48 of the tubing 36 is open to deliver fluid 22 into the patient 16 for insufflating or irrigating, while the tip 45 of the suction tube 35 is open to draw fluids 25 from within the patient 16. In some examples, the source of vacuum 52 draws the fluid 25 in series from the patient 16, through the open tip 45, through the suction tube 35, through the suction valve 15, through the umbilicus 32, and out through the control unit 30.

An operator 62 pressing their finger on the top end of the suction valve 15 places the suction valve 15 in a suction configuration to apply appreciable suction to the suction tube 35. The operator 62 removing their finger returns the suction valve 15 to a vented configuration, alleviating the vacuum in the suction tube 35. The suction valve 15 thus provides a simple way for turning the suction on and off at the tip 45 of the suction tube 35.

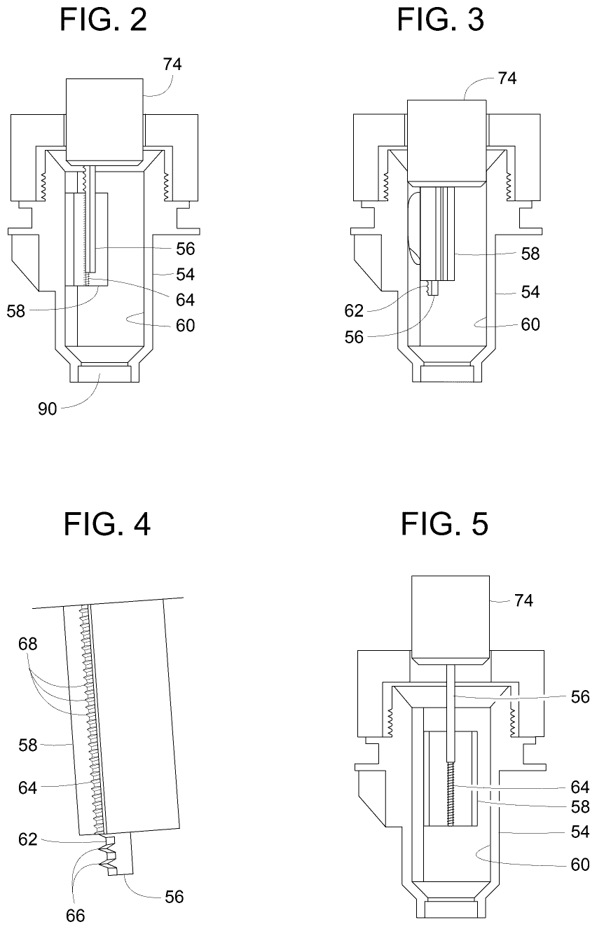

FIGS. 2-5 schematically illustrate some basic concepts for actuating the suction valve 15. In the illustrated examples, the suction valve 15 comprises a valve housing 54, a plunger 56, and a valve element 58. The valve element 58 rotates within a bore 60 of the valve housing 54 from a closed position to an open position in response to the plunger 56 being manually forced linearly from a home position (FIGS. 2 and 5) to a depressed position (FIG. 3). In some examples, the mechanical interaction between the plunger 56 and the valve element 58 is achieved by somewhat of a rack-and-pinion mechanism. In some examples, the plunger 56 includes a rack 62, and the valve element 58 includes somewhat of a pinion 64. In some examples, gear teeth 66 on the rack 62 mesh with gear teeth 68 on the pinion 64. In some examples, the pinion 64 is in the form of a screw, a threaded rod, or a threaded hole; wherein helical threads serve as the pinion's gear teeth 68. Some of the general concepts illustrated in FIGS. 2-5 can be adapted and applied to the more specific examples shown in FIGS. 1, and 6-24.

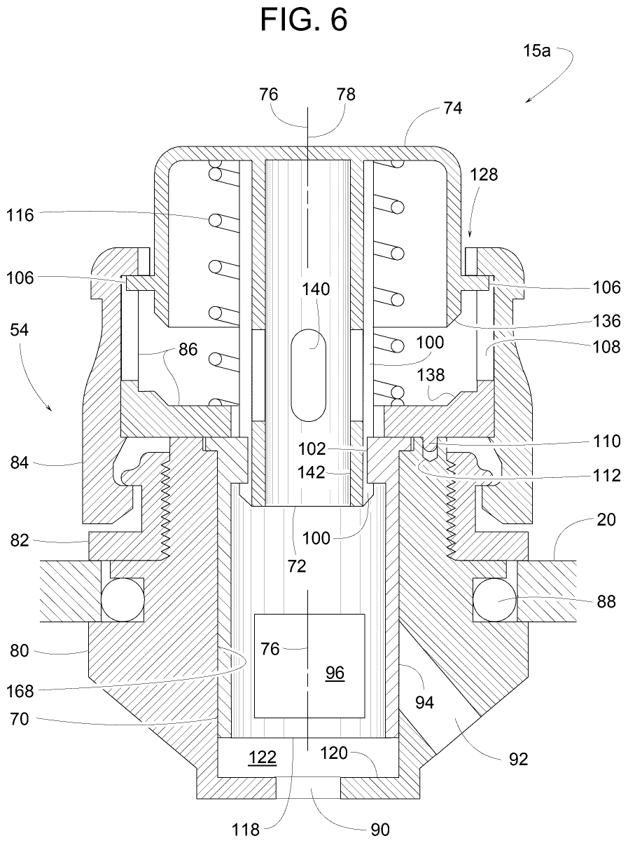

FIGS. 6-13 show the suction valve 15a, wherein FIGS. 6, 7 and 10 show the suction valve 15a in a vented configuration, and FIGS. 8 and 11 show the suction valve 15a in a suction configuration. In some examples, the suction valve 15a comprises the valve housing 54, a valve element 70, and a plunger 72 with a pushbutton 74. The suction valve 15a is normally closed so as not to apply suction at the tip 45 of the suction tube 35 when the suction valve 15a is at rest in the vented configuration.

To open the suction valve 15a and thus apply suction to the suction tube's tip 45, the operator 62 depresses the pushbutton 74 to force the plunger 72 farther into the valve housing 54, thereby placing the suction valve 15a in the suction configuration. Mechanical interaction between the plunger 72 and valve element 70 causes the valve element 70 to rotate about a first axis 76 from a closed position (FIGS. 6, 7 and 10) to an open position (FIGS. 8 and 11) in response to the plunger 72 moving in a linear direction along a second axis 78 from a home position (FIGS. 6, 7 and 10) to a depressed position (FIGS. 8 and 11) relative to the valve housing 54.

In some examples, the first axis 76 is substantially parallel to the second axis 78. The term, “substantially parallel” as it pertains to two axes means that the two axes lie within five degrees of each other. In some examples, the first axis 76 and the second axis 78 are collinear.

In some examples, the valve housing 54 is a single, monolithic piece. In other examples, the valve housing 54 is an assembly of multiple pieces. For instance, in some examples, the valve housing 54 comprises a base 80, a collar 82, a button holder 84, and a bushing 86. In some examples, the collar 82 screws onto the base 80 to fasten the valve housing 54 to the handle body 20. In some examples, a seal 88 (e.g., an O-ring) provides a sealed connection between the valve housing 54 and the handle body 20.

The valve housing 54, regardless of whether it is an assembly or a single piece, defines a first opening 90 and a second opening 92. In some examples, the first opening 90 is connected to the suction tube 35 in the flexible tubular probe 14, while the umbilicus 32 connects the second opening 92 to the source of vacuum 52. In other examples, the second opening 92 is connected to the suction tube 35 in the flexible tubular probe 14, while the umbilicus 32 connects the first opening 90 to the source of vacuum 52.

The rotational orientation of the valve element 70 within the valve housing 54 determines whether the first opening 90 is connected in fluid communication with the second opening 92, thus determining whether or not the suction tube 35 is connected in fluid communication with the source of suction 52. In some examples, the valve element 70 and a sealing portion 94 thereof are rotatable about the first axis 76 relative to the valve housing 54. In some examples, the valve element 70 and its sealing portion 94 are rotatable between the open and closed positions.

In the open position, an open window 96 defined by the valve element 70 is aligned with the second opening 92 to connect the second opening 92 in fluid communication with the first opening 90. A dashed line 98 shows the path of fluid communication between the first and second openings 90 and 92.

In the closed position, the sealing portion 94 covers the second opening 92 to close or at least significantly obstruct fluid flow through the second opening 92. The sealing portion 94 provides a greater obstruction to fluid communication between the first opening 90 and the second opening 92 when the sealing portion 94 is in the closed position than when the sealing portion 94 is in the open position. To enhance sealing between the valve housing 54 and the valve element 70, some examples of the valve element 70 have a compliant sealing material (e.g., silicone rubber, foam, flexible lip, etc.) around the perimeter of the open window 96 or around the second opening 92.

To rotate the valve element 70 in response to linear motion of the plunger 72, some examples of the plunger 72 include a first gear 100 (drive member), which meshes with a second gear 102 (driven member) of the valve element 70. The term, “gear” refers to any element with one or more teeth for meshing with one or more teeth of a second element such that movement of one element moves the other element. The peak of each gear tooth can be straight or angled with respect to the tooth's direction of travel. Some examples of gears include racks, pinions, internal ring gears, helical gears, screw gears, spur gears, bevel gears, and worm gears. In some examples, the first and second gears 100 and 102 are helical. In some examples, the peak edges of the first and second gears 100 and 102 lie at a helix angle 104 of about forty-five degrees. Such a helix angle 104 provides the valve element 70 with sufficient rotation for a given linear movement of the plunger 72. In other examples, however, the helix angle 104 is greater than forty-five degrees. In some examples, the helix angle 104 is less than forty-five degrees.

To ensure the valve element 70 is the primary rotating part, rather than the plunger 72, some examples of the suction valve 15a include an anti-rotation tab 106 (or multiple tabs) protruding radially outward from the pushbutton 74. The anti-rotation tab 106 is confined to travel within a slot 108 in the bushing 86. The tab 106 slides along the slot 108, as the plunger 72 moves linearly between the home and depressed positions. In some examples, the slot 108 is straight to prevent any rotation of the plunger 72. In other examples, the slot 108 is angled or slightly helical, so the plunger 72 itself experiences some rotation, but not necessarily as much as the valve element 70. Depending on the slot's helix angle (in examples where the slot 108 is angled), the plunger's degree and direction of rotation can contribute or subtract from the valve element's rotation.

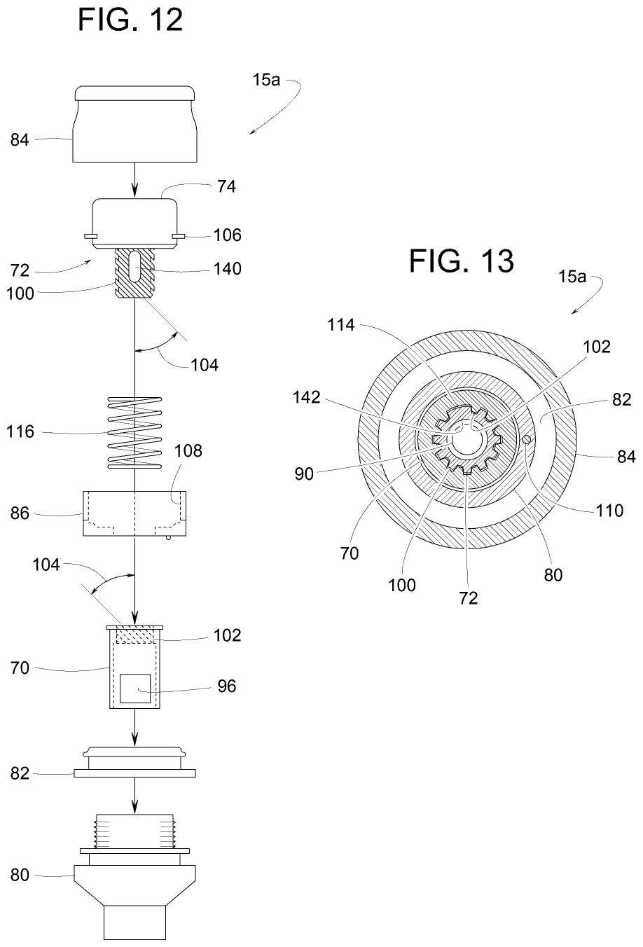

Upon assembling the suction valve 15a, as shown in FIG. 12, it is important to ensure a proper rotational relationship between the window 96 of the valve element 70 and the second opening 92 in the valve housing 54. To this end, some examples of the suction valve 15a include three alignment features. One feature is the tab 106 in the slot 108, which ensures proper rotational alignment between the plunger 72 and the bushing 86. A second feature is a key 110 in the bushing 86 that fits a cavity 112 in the valve housing 54. The second feature ensures proper rotational alignment between the bushing 86 and the valve housing 54. A third feature is an irregularity 114 (a key, an asymmetrical shape, etc.) at the meshing interface between the plunger 72 and the valve element 70, as shown in FIG. 13. The three features combined ensure proper rotational alignment of the valve housing's second opening 92 relative to the bushing 86 (second feature), the bushing 86 relative to the plunger 72 (first feature), the plunger 72 relative to the valve element 70 (third feature), and thus the valve element's window 96 and the valve housing's second opening 92.

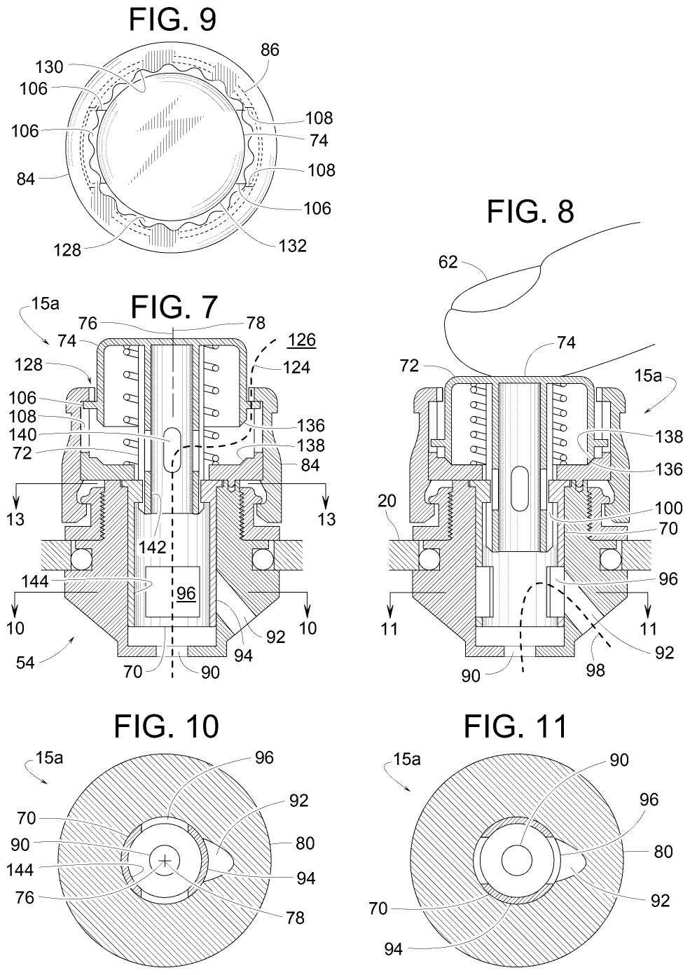

Some examples of the suction valve 15a include a spring 116 that urges the plunger 72 and its pushbutton 74 to the home position (FIGS. 6 and 7), so the suction valve 15a is normally closed when at rest. In the illustrated example, the spring 116 is a compression spring extending axially between the pushbutton 74 and the bushing 86. In other examples, the spring 116 is a compression spring extending axially between a bottom 118 of the valve element 70 and a bottom 120 of the valve housing 54. In some examples, the spring 116 is a torsion spring in an area 122 between the bottom 118 of the valve element 70 and the bottom 120 of the valve housing 54, wherein the torsion spring rotationally urges the valve element 70 to its closed position (FIGS. 6, 7 and 10), which in turn urges the plunger 72 to its home position.

The available area 122 for the spring 116 between the bottom 118 of the valve element 70 and the bottom 120 of the valve housing 54 is provided by having the valve element 70 be spaced apart from the second opening 90. The area 122 might also be beneficial as a dead space for incidental tissue fragments to collect without interfering with the operation of the suction valve 15a.

Some examples of the plunger 72 include a vent passageway 124 (dashed line) connecting the first opening 90 in fluid communication with a surrounding atmosphere 126 when the plunger 70 is in the home position. The term, “surrounding atmosphere” refers to the room air to which the exterior of the suction valve 15a is exposed. In some examples, air from the surrounding atmosphere 126 can enter the suction valve 15a through an annular gap 128 between a serrated inner edge 130 of the button holder 84 and an outer diameter 132 of the pushbutton 74. The first opening 90 being vented to atmosphere 126 ensures virtually no suction reaches the suction tube's tip 45 when the plunger 72 is in the home position (FIG. 7).

In FIG. 7, the dashed line representing the vent passageway 124 shows the path of fluid communication between the first opening 90 and the surrounding atmosphere 126 when the pushbutton 74 is in the home position. As illustrated by the dashed line in FIG. 7, atmospheric air 126 can pass in series through the annular gap 128, between a valve plug 136 of the plunger 72 and a valve seat 138 of the bushing 86, through an opening 140 in the plunger 72, through an inner bore 142 of the plunger 72, through an inner bore 144 of the valve element 70, past the bottom 118 of the valve element 70, and to the first opening 90.

When the pushbutton 74 is in the closed position (FIG. 8), the valve plug 136 seals against the valve seat 138. This prevents atmospheric air 126 from flowing freely into the plunger's inner bore 142, and thus prevents the first and second openings 90 and 92 from being vented to atmosphere 126. Consequently, suction can be applied to the suction tube's tip 45. In some examples, the valve seat 138 and valve plug 136 have beveled edges that are conducive for carefully metering airflow.

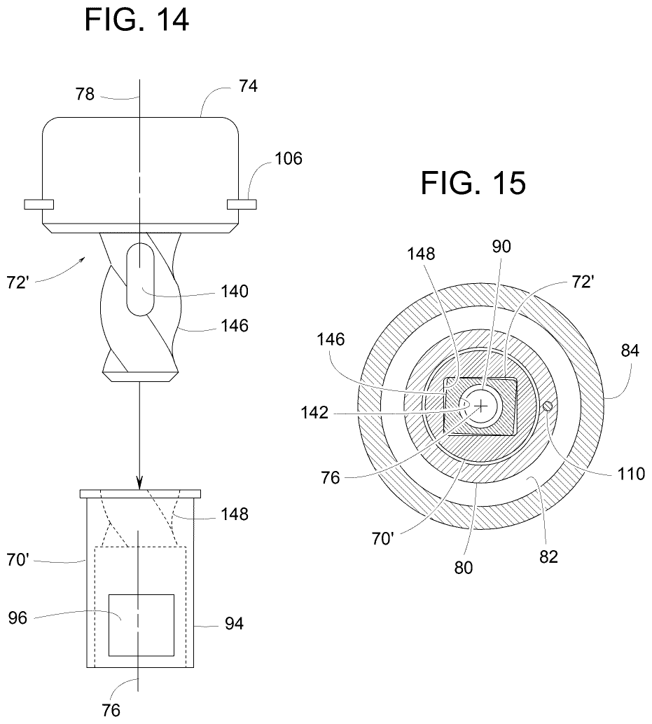

FIGS. 14 and 15 show an alternate plunger 72′ and valve element 70′ for use in the suction valve 15a. The plunger 72′ includes a helical portion 146 extending through a helical bore 148 defined by the valve element 70′. In a manner like plunger 72 and valve element 70 of FIGS. 6-12, the valve element 70′ rotates about the first axis 76 from its closed position to its open position in response to the plunger 72′ moving from its home position to its depressed position.

In some examples, the cross-sectional areas of the plunger 72′ and valve element 70′ are rectangular, as shown in FIG. 15. The rectangular shapes ensure that the plunger 72′ and the valve element 70′ are assembled in their proper rotational orientation. With a rectangular shape, the plunger 72′ and the valve element 70′ can be assembled in two ways. They can be assembled as shown in FIG. 15 or assembled 180 degrees of that. In either case, one of the two windows 96 will properly align with the second opening 92 when the valve element 70′ is in the open position.

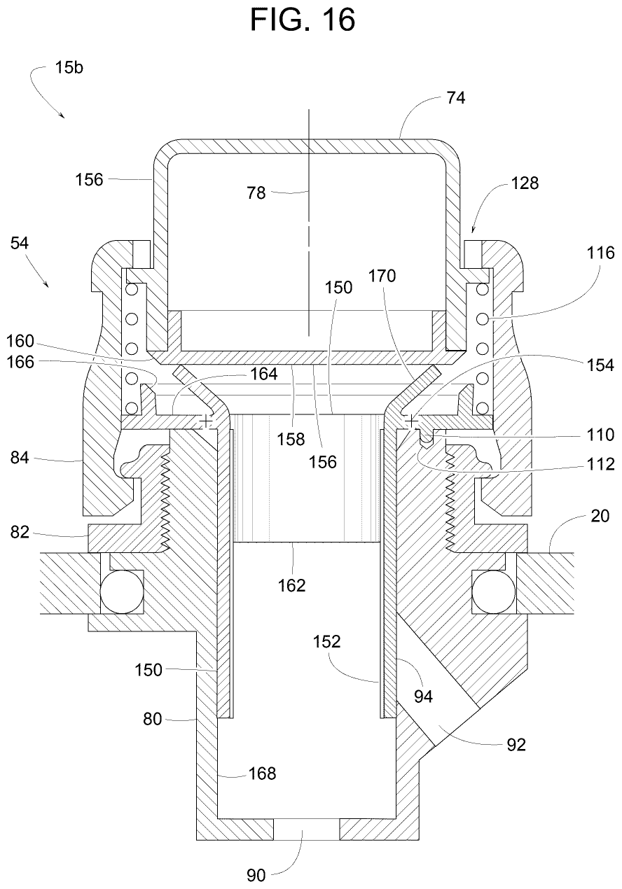

FIGS. 16-20 show the suction valve 15b, wherein FIGS. 16, 17, 21 and 23 show the suction valve 15b in a vented configuration, and FIGS. 18, 22 and 24 show suction valve 15b in a suction configuration. Suction valve 15a and 15b are similar in that they both have a valve element that rotates in response to linear movement of a plunger. In contrast, however, the suction valve 15b comprises a valve element 150 with a flap 152 (sealing portion) that rotates by deflection about a first axis 154 that is substantially perpendicular to the second axis 78 of a plunger 156. The term, “substantially perpendicular” as it pertains to two axis means that the two axes are within five degrees of being perpendicular to each other. In response to the plunger 156 being moved linearly along the second axis 78 from a home position (FIGS. 16, 17, 21 and 23) to a depressed position (FIGS. 18, 22 and 24), the valve element's flap 152 deflects from a closed position (FIGS. 16, 17, 21 and 23) to an open position (FIGS. 18, 22 and 24) relative to a valve housing 54.

In this example, the valve housing 54 does not include the bushing 86. The plunger 156, in this example, comprises the pushbutton 74 and a bottom 158 with a valve plug 160.

The valve element 150 comprises a ring 162 (e.g., a hollow cylinder), a flange 164, the flap 152 (sealing portion), a valve seat 166, and the key 110. The key 110 fits into the valve housing's cavity 112 to ensure that the valve element's flap 152 is in the proper rotational orientation relative to the second opening 92 in the valve housing 54. The ring 162 fits into a bore 168 of the valve housing 54 to establish proper radial alignment between the valve element 150 and valve housing's bore 168.

In some examples, the valve element 150 is a monolithic structure, wherein a lever 170 and the flap 152 are a seamless integral portion of the valve element 150. In some examples, the valve element 150 is made of a sufficiently resilient polymer. The term, “sufficiently resilient” refers to a material property that enables at least some portions of the valve element 150 to elastically and restorably bend between the valve element's open and closed positions. Some example polymeric materials of the valve element 150 include nylon, PTFE (polytetrafluoroethylene), PEEK (polyetheretherketone), polycarbonate, ABS (acrylonitrile-butadiene-styrene), HDPE (high-density polyethylene), UHMW (ultra-high molecular-weight polyethylene), POM (polyoxymethylene, polyacetal, Delrin, Celcon, etc.), POM-C (polyoxymethylene copolymer), and POM-H (polyoxymethylene homopolymer).

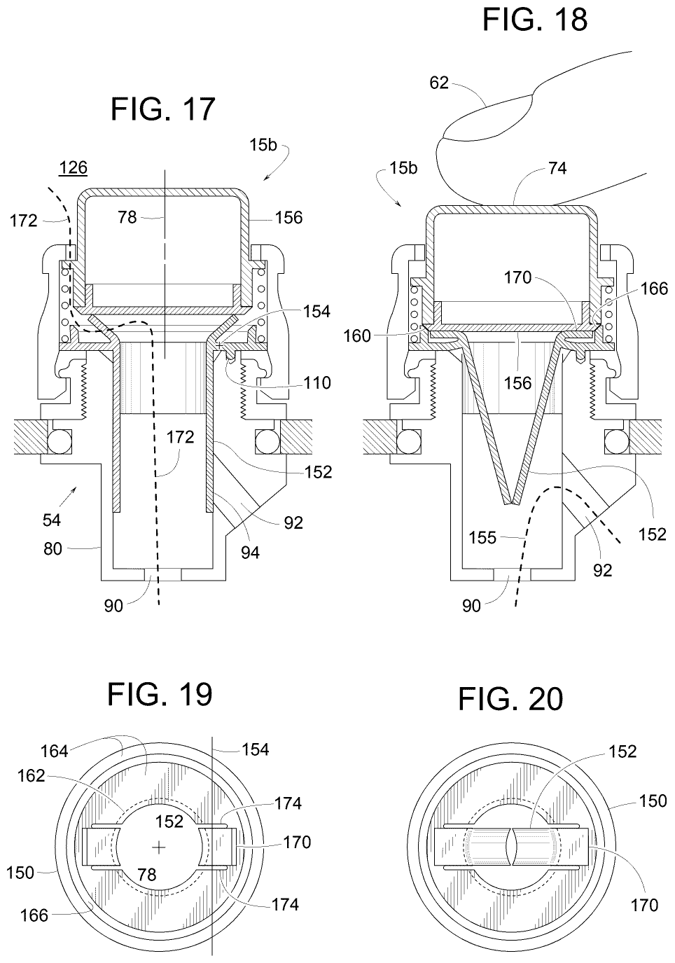

To open the suction valve 15b to the suction configuration, the operator 62 depresses the plunger's pushbutton 74, as shown in FIG. 18. Upon doing so, the plunger's valve plug 160 seals down against the valve seat 166 to close a vent path 172 between the first opening 90 and the surrounding atmosphere 126. Depressing the pushbutton 74 also forces the bottom 158 of the plunger 156 to bend the lever 170 down toward the flange 164, which tilts the flap 152 away from the second opening 92 in the valve housing 54. FIG. 19 shows a top view of the valve element 150 when the suction valve 15b is in its vented configuration, as shown in FIG. 17. FIG. 20 shows a top view of the valve element 150 when the suction valve 15b is in the suction configuration, as shown in FIG. 18.

Slits 174 in the valve element 150 provide the flap 152 and the lever 170 with the freedom to rotate by pivoting as a teeter-totter with the first axis 154 as a fulcrum. The flap 152 uncovering the second opening 92 establishes a fluid communication path 155 between the first and second openings 90 and 92, and thus delivers suction to the suction tube's tip 45.

In some examples, the valve element 150 includes two or more flaps 152 and levers 170, which provide a more even, balanced distribution of axial forces up against the underside of the plunger's bottom 158. In some examples, the compression spring 116 urges the plunger's pushbutton 74 to its home position.

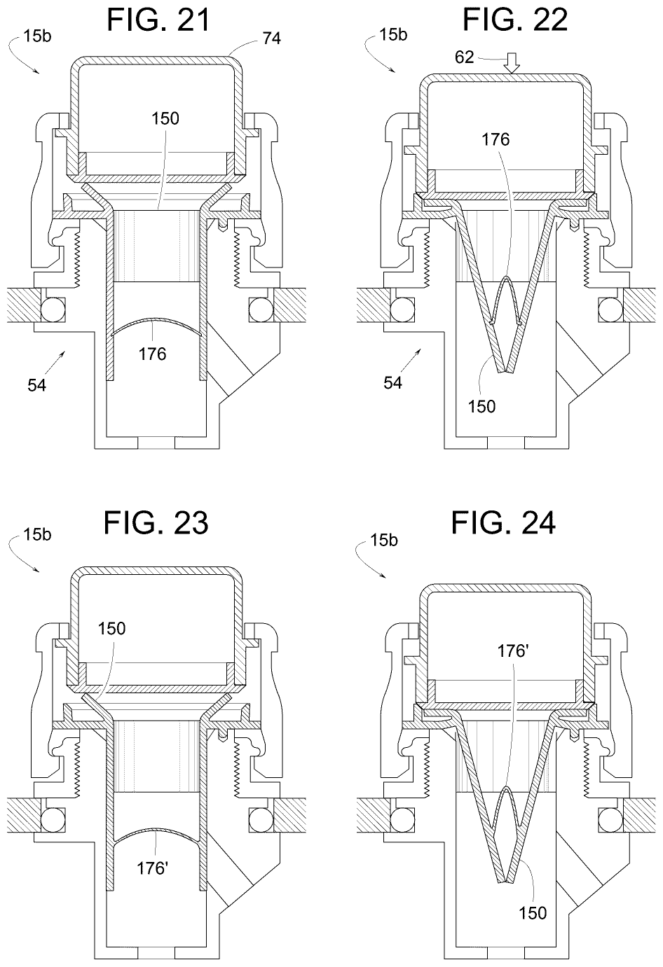

In addition, or alternatively, the resilience of the lever 170 and flaps 152 is what urges the plunger's pushbutton 74 back to the home position, so in some examples, the compression spring 116 is omitted, as shown in FIGS. 21-24. In some examples, a leaf spring 176 (e.g., made of spring steel) urges the flaps 152 to their closed position and urges the plunger's pushbutton 74 to the home position. In some examples, the valve element 150 includes an integral leaf spring 176′ for the same purpose. In the example of the integral leaf spring 176′, the entire valve element 150, including the leaf spring 176′, is a seamless, unitary monolithic piece.

1. A suction valve for controlling a fluid flowing through an endoscope that includes a flexible tubular probe extending from a handle body, the endoscope being connectable to a suction source, the suction valve comprising:
a valve housing to be supported by the handle body, the valve housing defining a first opening and a second opening;
a valve element within the valve housing, the valve element including a sealing portion that is rotatable about a first axis between an open position and a closed position relative to the valve housing; and
a plunger engaging the valve element and being movable relative thereto, the plunger being further movable linearly along a second axis between a home position and a depressed position relative to the valve housing, the sealing portion moving from the closed position to the open position in response to the plunger moving from the home position to the depressed position, the sealing portion providing a greater obstruction to fluid communication between the first opening and the second opening when the sealing portion is in the closed position than when the sealing portion is in the open position.

2. The suction valve of claim 1, wherein the valve element is spaced apart from the first opening when the sealing portion is in the closed position, the valve element is spaced apart from the second opening when the sealing portion is in the open position, the valve element covers the second opening when the sealing portion is in the closed position, and the valve element uncovers the second opening when the sealing portion is in the open position.

3. The suction valve of claim 1, wherein the first axis is substantially parallel with the second axis.

4. The suction valve of claim 1, wherein the first axis is collinear with the second axis.

5. The suction valve of claim 1, wherein the plunger includes a gear.

6. The suction valve of claim 1, wherein the plunger includes a first gear, the valve element includes a second gear, and the first gear meshes with the second gear.

7. The suction valve of claim 1, wherein the plunger includes a helical portion extending through a helical bore defined by the valve element.

8. The suction valve of claim 1, wherein the plunger defines a vent passageway connecting the first opening in fluid communication with a surrounding atmosphere when the plunger is in the home position.

9. The suction valve of claim 1, wherein the plunger includes a valve plug, the valve housing includes a valve seat, the valve plug engages the valve seat when the plunger is in the depressed position, and the valve plug is spaced apart from the valve seat when the plunger is in the home position to define a vent passageway connecting the first opening in fluid communication with a surrounding atmosphere.

10. The suction valve of claim 1, wherein the first axis is substantially perpendicular to the second axis.

11. The suction valve of claim 1, wherein the first axis is substantially perpendicular to the second axis, the valve element includes lever and a flap, the sealing portion of the valve element is on the flap, the lever engages the plunger, the flap rotates by pivoting about the first axis in response to the plunger pushing against the lever as the plunger moves along the second axis from the home position to the depressed position.

12. The suction valve of claim 11, wherein the lever and the flap are a seamless integral portion of the valve element.

13. The suction valve of claim 12, wherein the seamless integral portion resiliently deflects in response to the plunger moving from the home position to the depressed position.

14. The suction valve of claim 12, wherein the seamless integral portion resiliently deflects in response to the plunger moving from the home position to the depressed position, and the lever urges the plunger to the home position.

15. The suction valve of claim 1, wherein the first opening is in fluid communication with the suction source when the endoscope is connected to the suction source, and the second opening is in fluid communication with the flexible tubular probe.

16. The suction valve of claim 1, wherein the second opening is in fluid communication with the suction source when the endoscope is connected to the suction source, and the first opening is in fluid communication with the flexible tubular probe.

17. A suction valve for controlling a fluid flowing through an endoscope that includes a flexible tubular probe extending from a handle body, the endoscope being connectable to a suction source, the suction valve comprising:
a valve housing to be supported by the handle body, the valve housing defining a first opening and a second opening;
a valve element within the valve housing, the valve element including a sealing portion that is rotatable about a first axis between an open position and a closed position relative to the valve housing, the valve element defining a bore encircling the first axis; and
a plunger extending at least partially through the bore of the valve element, the plunger being movable linearly along a second axis between a home position and a depressed position relative to the valve housing, the second axis being substantially parallel with the first axis, the sealing portion rotating about the first axis from the closed position to the open position in response to the plunger moving from the home position to the depressed position, the sealing portion providing a greater obstruction to fluid communication between the first opening and the second opening when the sealing portion is in the closed position than when the sealing portion is in the open position.

18. The suction valve of claim 17, wherein the plunger includes a helical portion extending through and mating with the bore of the valve element.

19. A suction valve for controlling a fluid flowing through an endoscope that includes a flexible tubular probe extending from a handle body, the endoscope being connectable to a suction source, the suction valve comprising:
a valve housing to be supported by the handle body, the valve housing defining a first opening and a second opening;
a valve element within the valve housing, the valve element comprising a lever and a flap with a sealing portion on the flap, the lever and the flap are a seamless integral portion of the valve element, the sealing portion is rotatable about a first axis between an open position and a closed position relative to the valve housing; and
a plunger engaging the lever and being movable linearly along a second axis between a home position and a depressed position relative to the valve housing, the second axis being substantially perpendicular to the first axis, the valve element pivoting by resilient deflection about the first axis from the closed position to the open position in response to the plunger moving along the second axis from the home position to the depressed position, and the sealing portion providing a greater obstruction to fluid communication between the first opening and the second opening when the sealing portion is in the closed position than when the sealing portion is in the open position.

20. The suction valve of claim 19, wherein the valve element is spaced apart from the first opening when the sealing portion is in the closed position, the valve element is spaced apart from the second opening when the sealing portion is in the open position, the valve element covers the second opening when the sealing portion is in the closed position, the valve element uncovers the second opening when the sealing portion is in the open position, and the lever urges the plunger to the home position.
